(19) United States
(12) Patent Application Publication
(10) Pub. No.: US 2015/0313937 A1
(43) Pub. Date: Nov. 5, 2015

(54) Solution And Method For Treating Cancer

(71) Applicant: Priscilla May Miller, West Des Moines, IA (US)

(72) Inventor: Priscilla May Miller, West Des Moines, IA (US)

(21) Appl. No.: 14/269,185

(22) Filed: May 4, 2014

(51) Int. Cl. A61K 33/20; A61K 31/265; A61K 9/00; A61K 31/196; A61K 31/20; A61K 31/10; A61K 31/7076

(52) U.S. Cl. CPC A61K 33/20; A61K 31/10; A61K 31/265; A61K 31/7076; A61K 31/196; A61K 31/20; A61K 9/0019

(57) ABSTRACT

Cancer has been a persistent, serious health problem for several decades. The present invention relates to a chemical solution of relatively common chemicals that can be mixed and injected into a patient to destroy existing cancer cells or to immunize people from cancer. The solution is mixed using four distinct vials holding different chemicals according to a certain protocol. The resulting solution is injected into a patient according to a specific dosing regimen.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not Applicable

### BACKGROUND OF THE INVENTION

1. Field of the Invention

Cancer has been a persistent, serious health problem for several decades now. While treatment methods have improved, cancer continues to affect millions of lives every year. Additionally, existing treatments typically involve either surgery for removing a cancerous growth, or chemotherapy to destroy cancer cells that cannot be operated on. These methods can be invasive to the body, and also potentially create serious side effects for the patient. The present invention allows for a much less intrusive method for treating all forms of cancer.

2. Description of Related Art

While there are many patents and publications that are intended to treat and cure cancer, none use the precise combination of treatment solution and treatment procedure of the present invention.

### BRIEF SUMMARY OF THE INVENTION

The present invention relates to a process for treating cancer. A solution is created from existing chemicals and the solution is injected into a patient to destroy existing cancer cells. The patient is injected based on a fixed dosing regimen. After the dosing regimen is completed, the patient is observed for a set time period. If cancer cells still exist after the observation period, the dosing regimen is repeated.

### BRIEF DESCRIPTION OF THE SEVERAL VIEWS OF THE DRAWINGS

The invention is a process for the treatment of cancer with the steps of the process illustrated in the accompanying drawings, which form a part hereof and wherein:

FIG. 1 is a flowchart depicting the steps in the overall process of the treatment.

FIG. 2 is a flowchart depicting the steps involved in creating the treating mixture.

### DETAILED DESCRIPTION OF THE INVENTION

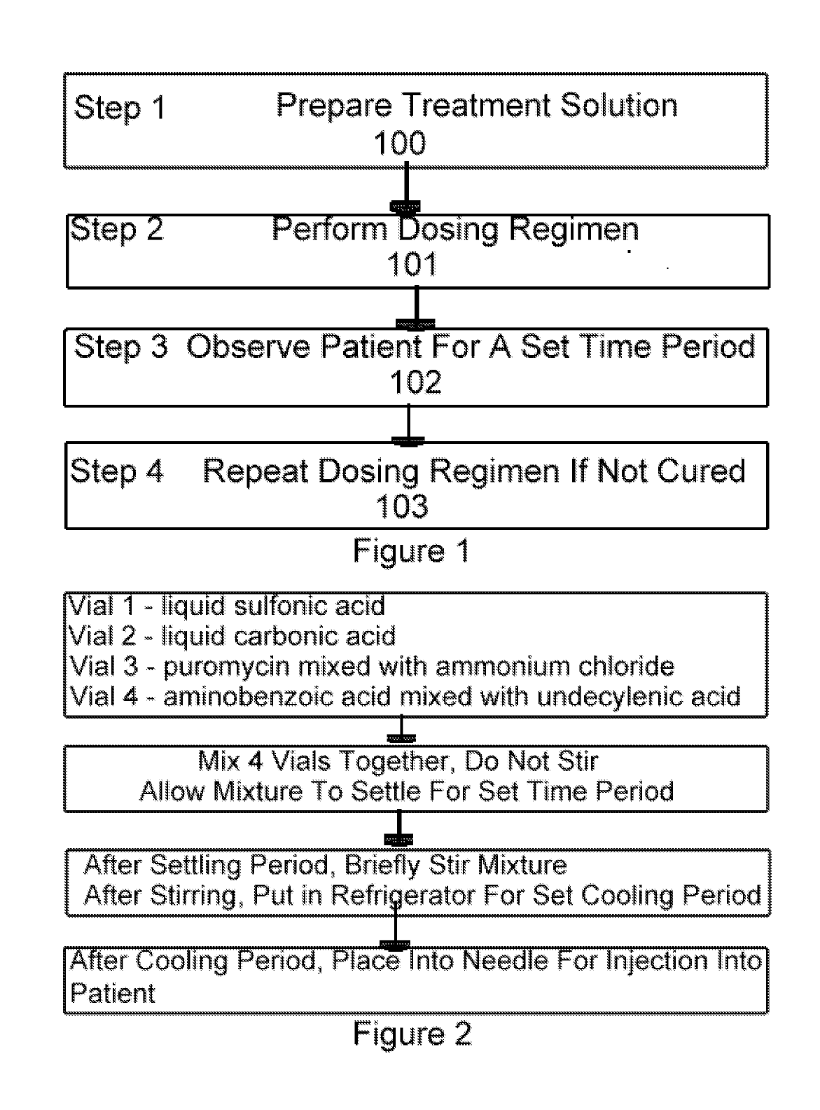

The invention is carried out as illustrated in FIGS. 1-2. This invention is used to treat all forms of cancer in a less intrusive manner for the patient. The first step of the process 100 is to create the solution that will be injected into a patient to destroy existing cancer cells. To prepare this treatment solution 100, requires a specific combination of chemicals mixed using a specific set of steps.

Four vials holding four distinct chemical solutions will be mixed together to create the treating mixture. The first vial will preferably hold a liquid sulfonic acid. A sulfonic acid substitute that has very similar chemical properties could potentially be used, but sulfonic acid itself would be the most effective first ingredient.

The second vial will preferably hold a liquid carbonic acid. A carbonic acid substitute that has very similar chemical properties could potentially be used, but carbonic acid itself would be the most effective second ingredient.

The third vial will preferably hold a liquid mixture of puromycin and ammonium chloride. Substitutes that have very similar chemical properties to either puromycin or ammonium chloride could potentially be used, but puromycin and ammonium chloride themselves would be the most effective ingredients to form this mixture for the third ingredient.

The fourth vial will preferably hold a liquid mixture of aminobenzoic acid and undecylenic acid. Substitutes that have very similar chemical properties to either aminobenzoic acid or undecylenic acid could potentially be used, but aminobenzoic acid and undecylenic acid themselves would be the most effective ingredients to form this mixture for the fourth ingredient.

After preparing the four vials with their corresponding ingredients, the four vials are mixed together. It is critical that upon mixture, the solution is not stirred. After mixing the vials, the resulting solution is allowed to settle for a certain time period. The preferred time period for this settling is ten days.

After the solution has been allowed to settle, the solution will be briefly stirred, and then placed into a refrigerator for a set cooling period. The preferred time period for this cooling period is three days. After the cooling period, the solution is ready to be injected into the patient.

When injecting the patient with the solution, a fixed dosing regimen 101 is followed. This preferred dosing regimen 101 requires the patient to be injected the solution once a week for four weeks. The dosing regimen requires a dose of 1 cubic centimeter of the solution the first week, 2 cubic centimeters the second week, 2.5 cubic centimeters the third week and 3 cubic centimeters the fourth week. After this dosing regimen 101 has been completed, the cancer cells in the patient's body should be destroyed. To confirm this, the patient is observed for a fixed observation period 102.

This observation period 102 is preferably for a period of six weeks. Multiple checkups can be performed within this six week period to determine whether cancer cells still exist, or whether new cancer cells exist within the patient. If it is determined that the cancer has not been cured, the treatment process 103 is begun again by following the same dosing regimen 101.

1. A method for destroying cancer cells or preventing cancer cells from growing, comprising:
a first vial comprising an effective amount of a liquid sulfonic acid,
a second vial comprising an effective amount of a liquid carbonic acid,
a third vial comprising a liquid mixture comprised of an effective amount of puromycin and an effective amount of ammonium chloride;
a fourth vial comprising a mixture of an effective amount of liquid aminobenzoic acid and an effective amount of liquid undecylenic acid;
wherein said first vial, said second vial, said third vial and said fourth vial are combined together into a single combination mixture in a single large vial;
wherein said single combination mixture is not stirred when combining said first vial, said second vial, said third vial and said fourth vial and said single combination mixture is allowed to set for a setting period;
wherein after said setting period said single combination mixture is stirred and put into a refrigerator for a cooling period;
wherein after said cooling period said single combination mixture is put into a needle to be injected into a patient;
wherein said single combination mixture is injected into a patient by a dosing regimen.

2. The method for destroying cancer cells or preventing cancer cells from growing of claim 1, wherein said setting period is ten days.

3. The method for destroying cancer cells or preventing cancer cells from growing of claim 2, wherein said cooling period is three days.

4. The method for destroying cancer cells or preventing cancer cells from growing of claim 3, wherein said set dosing regimen consists of injecting a patient once a week for four weeks, further comprising injecting said patient with 1 cubic centimeter of said single combination mixture in a first week of said set dosing regimen, injecting said patient with 2 cubic centimeters of said single combination mixture in a second week of said dosing regimen, injecting said patient with 2.5 cubic centimeters of said single combination mixture in a third week of said dosing regimen, injecting said patient with 3 cubic centimeters of said single combination mixture in a fourth week of said dosing regimen.

5. The method for destroying cancer cells or preventing cancer cells from growing of claim 4, wherein said patient is monitored for an observation period after said dosing regimen.

6. The method for destroying cancer cells or preventing cancer cells from growing of claim 5, wherein said observation period is six weeks.

7. The method for destroying cancer cells or preventing cancer cells from growing of claim 6, wherein said dosing regimen is repeated if cancer cells are not destroyed after said observation period.

8. A method for destroying cancer cells or preventing cancer cells from growing, comprising:
a first vial comprising an effective amount of a liquid sulfonic acid,
a second vial comprising an effective amount of a liquid carbonic acid,
a third vial comprising a mixture comprised of an effective amount of puromycin and an effective amount of ammonium chloride;
a fourth vial comprising a mixture of a liquid aminobenzoic acid and a liquid undecylenic acid;
wherein said first vial, said second vial, said third vial and said fourth vial are combined together into a single combination mixture in a single large vial;
wherein said single combination mixture is not stirred when combining said first vial, said second vial, said third vial and said fourth vial and said single combination mixture is allowed to set for a setting period;
wherein said single combination mixture is stirred and put into a refrigerator for a cooling period;
wherein after said cooling period said single combination mixture is put into a needle to be injected into a patient;
wherein said single combination mixture is injected into a patient by a dosing regimen;
wherein said patient is monitored for an observation period after performing said dosing regimen;
wherein said patient receives said dosing regimen again if cancer cells still exist after said observation period.

9. The method for destroying cancer cells or preventing cancer cells from growing of claim 8, wherein said first vial comprises a liquid sulfonic acid or liquid sulfonic acid substitute.

10. The method for destroying cancer cells or preventing cancer cells from growing of claim 9, wherein said second vial comprises a liquid carbonic acid or liquid carbonic acid substitute.

11. The method for destroying cancer cells or preventing cancer cells from growing of claim 10, wherein said third vial comprises a liquid mixture comprised of an effective amount of puromycin or puromycin substitute and an effective amount of ammonium chloride or ammonium chloride substitute.

12. The method for destroying cancer cells or preventing cancer cells from growing of claim 11, wherein said fourth vial comprises a liquid undecylenic acid or liquid undecylenic acid substitute.

13. The method for destroying cancer cells or preventing cancer cells from growing of claim 12, wherein said setting period is ten days.

14. The method for destroying cancer cells or preventing cancer cells from growing of claim 13, wherein said cooling period is three days.

15. The method for destroying cancer cells or preventing cancer cells from growing of claim 14, wherein said set dosing regimen consists of injecting a patient once a week for four weeks, further comprising injecting said patient with 1 cubic centimeter of said single combination mixture in a first week of said set dosing regimen, injecting said patient with 2 cubic centimeters of said single combination mixture in a second week of said dosing regimen, injecting said patient with 2.5 cubic centimeters of said single combination mixture in a third week of said dosing regimen, injecting said patient with 3 cubic centimeters of said single combination mixture in a fourth week of said dosing regimen.

16. The method for destroying cancer cells or preventing cancer cells from growing of claim 15, wherein said patient is monitored for an observation period after said dosing regimen.

17. The method for destroying cancer cells or preventing cancer cells from growing of claim 16, wherein said observation period is six weeks.

18. A method for destroying cancer cells or preventing cancer cells from growing, comprising:
a first vial comprising an effective amount of a liquid sulfonic acid,
a second vial comprising an effective amount of a liquid carbonic acid,
a third vial comprising a liquid mixture comprised of an effective amount of puromycin and an effective amount of ammonium chloride;
a fourth vial comprising a mixture of a liquid aminobenzoic acid and a liquid undecylenic acid;
wherein said first vial, said second vial, said third vial and said fourth vial are combined together into a single combination mixture in a single large vial;
wherein said single combination mixture is not stirred when combining said first vial, said second vial, said third vial and said fourth vial and said single combination mixture is allowed to set for a setting period of ten days;
wherein said single combination mixture is stirred and put into a refrigerator for a cooling period of three days;
wherein after said cooling period said single combination mixture is put into a needle to be injected into a patient;
wherein said single combination mixture is injected into a patient by a dosing regimen;
wherein said dosing regimen consists of one injection a week for four weeks,
further comprising injecting said patient with 1 cubic centimeter of said single combination mixture in a first week of said set dosing regimen, injecting said patient with 2 cubic centimeters of said single combination mixture in a second week of said dosing regimen,
injecting said patient with 2.5 cubic centimeters of said single combination mixture in a third week of said dosing regimen, injecting said patient with 3 cubic centimeters of said single combination mixture in a fourth week of said dosing regimen;
wherein said patient is monitored for an observation period of six weeks;
wherein said patient receives said dosing regimen again if cancer cells still exist after said observation period.

19. The method for destroying cancer cells or preventing cancer cells from growing of claim 18, wherein said first vial comprises a liquid sulfonic acid or liquid sulfonic acid substitute;
wherein said second vial comprises a liquid carbonic acid or liquid carbonic acid substitute.

20. The method for destroying cancer cells or preventing cancer cells from growing of claim 19, wherein said third vial comprises an effective amount of puromycin or puromycin substitutue mixed with an effective amount of ammonium chloride or ammonium chloride substitute;
wherein said fourth vial comprises a liquid aminobenzoic acid or liquid aminobenzoic acid substitute mixed with a liquid undecylenic acid or liquid undecylenic acid substitute.
